(19) United States
(12) Patent Application Publication
(10) Pub. No.: US 2026/0108395 A1
(43) Pub. Date: Apr. 23, 2026

(54) ABSORBENT PENILE ENCLOSURE

(71) Applicant: Bebi Shelliza Ballee Goolsarran, Miramar, FL (US)

(72) Inventor: Bebi Shelliza Ballee Goolsarran, Miramar, FL (US)

(21) Appl. No.: 19/424,442

(22) Filed: Dec. 18, 2025

Related U.S. Application Data
(63) Continuation-in-part of application No. 18/235,648, filed on Aug. 18, 2023.

(51) Int. Cl. A61F 13/471; A61F 13/15; A61F 13/45; A61F 13/511; A61F 13/514; A61F 13/58

(52) U.S. Cl. CPC A61F 13/471; A61F 13/15203; A61F 13/51113; A61F 13/51121; A61F 13/51401; A61F 13/581; A61F 2013/15121; A61F 2013/15146; A61F 2013/15373; A61F 2013/4506; A61F 2013/583

(57) ABSTRACT

An absorbent penile enclosure that includes a unitary body with an absorbent layup including an inner layer of a permeable material fabric material, including an intermediate superimposed over the inner layer and of a fabric absorbent material, including an exterior layer superimposed over and enclosing the intermediate layer and of an impervious material and that is configured with a base portion defined by two opposing outer side edges of the unitary body, an outer lower edge, and an outer upper edge, having the absorbent layup thereon spanning along the base portion, and having a base fastener disposed proximal to one of the two opposing outer side edges and with two auxiliary coupling members each extending outwardly from the outer upper edge of the base portion, disposed in a cantilevered configuration with a distal edge, having the absorbent layup thereon, and having an auxiliary fastener disposed proximal to the distal edge.

### FIELD OF THE INVENTION

The present invention relates generally to an absorbent article for enclosing a male's genitalia and, more particularly, relates to selectively couplable enclosure or pad for a male's penis that is configured to absorb urine or other bodily fluids emitted therefrom.

### BACKGROUND OF THE INVENTION

Incontinence is a very common issue prevalent among many male individuals. Even if a male user is not incontinent, inadvertent urine leakage is an issue many male users experience. These issues are extremely problematic due to the adverse effects on an individual's clothing, in addition to the emotional effects they cause to the individual, personally. Some known penile enclosures directed toward absorbing urine include forming a cylindrical enclosure with an absorbent material, e.g., cotton, with a waterproof layer surrounding said absorbent material. These penile enclosures, while effective for absorbing urine, problematically leave a significant amount of moisture in direct contact with the user's skin. This moisture also increases the likelihood of the enclosure being inadvertently removed. Notwithstanding the moisture in direct contact with the user's skin, these penile enclosures also become routinely and inadvertently removed simply because of their structural configuration and attachment to the user.

To that end, some known penile enclosures have been created to more securely fasten to the user, including using elastic bands, hook-and-loop couplers, or adhesive. Each of these known enclosures are problematic in that they only temporarily secure the enclosure to the user, do not adequately secure the enclosure to the user when the user is more active, and/or over-designed causing them to be comparatively costly, difficult for consumers to figure out how to utilize, and time consuming to attach and detach from the user.

Therefore, a need exists to overcome the problems with the prior art as discussed above.

### SUMMARY OF THE INVENTION

The invention provides an absorbent penile enclosure that overcomes the hereinafore-mentioned disadvantages of the heretofore-known devices and methods of this general type and that effectively absorbs and retains urine in the enclosure for disposal and/or enables effective, comfortable, and safe coupling of catheter or other device for removal of urine. Furthermore, the present invention ensures a secure attachment of the enclosure to the user, even while more active.

With the foregoing and other objects in view, there is provided, in accordance with the invention, an absorbent penile enclosure is disclosed that includes an absorbent penile enclosure a unitary body with an absorbent layup including an inner layer of a permeable material fabric material, including an intermediate superimposed over the inner layer and of a fabric absorbent material, including an exterior layer superimposed over and enclosing the intermediate layer and of an impervious material, that is configured with a base portion defined by two opposing outer side edges of the unitary body, an outer lower edge, and an outer upper edge, having the absorbent layup thereon spanning along the base portion for surrounding a user's penis, and has at least one base fastener disposed proximal to one of the two opposing outer side edges. The body is also configured with two auxiliary coupling members each extending outwardly from the outer upper edge of the base portion, is disposed in a cantilevered configuration with a distal edge, has the absorbent layup thereon, and has at least one auxiliary fastener disposed proximal to the distal edge.

In accordance with a further feature of the present invention, the intermediate layer is encapsulated by the inner and exterior layers.

In accordance with a further feature of the present invention, an exterior layer thickness of the exterior layer is less than an intermediate layer thickness of the intermediate layer and the intermediate layer thickness is greater than the inner layer thickness of the inner layer.

In accordance with a further feature of the present invention, the at least one base fastener is disposed on the inner layer and is configured to directly couple with a fastener disposed on the exterior layer.

In accordance with another feature, an embodiment of the present invention includes the two auxiliary coupling members each having outer side edges, wherein the base portion is configured with a first offset length separating one of the two opposing outer side edges and one of the outer side edges of the two auxiliary coupling members that is greater than a second offset length separating another of the two opposing outer side edges and another of the outer side edges of the two auxiliary coupling members.

In accordance with an additional feature of the present invention, the at least one auxiliary fastener is disposed on the inner layer and is configured to directly couple with a fastener disposed on the exterior layer on the base portion.

In accordance with an exemplary feature of the present invention, the at least one auxiliary fastener is disposed on the inner layer and is configured to directly couple with a fastener disposed on the exterior layer on the base portion.

In accordance with yet another feature, an embodiment of the present invention also includes each of the two auxiliary coupling members having inner side edges opposing one of the outer side edges thereon, wherein the inner side edges define a channel spanning from a joint formed by the two auxiliary coupling members to the distal edges thereon.

In accordance with another feature of the present invention, the channel is less than 1 inch spanning from the joint to the distal edges on each of the two auxiliary coupling members.

In accordance with a further feature, an embodiment of the present invention also includes a catheter tube disposed in the channel and configured to remove liquid from a user's penis out through the unitary body.

In accordance with a further feature of the present invention, the inner layer is infused with an aloe material, the inner layer is made of a polyester fiber material, the exterior layer is of a polyethylene material, the inner layer is of a moisture wicking material, and the inner layer and the intermediate layer are of a thickness less than approximately 3 mm.

Although the invention is illustrated and described herein as embodied in an absorbent penile disclosure, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic of the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the enclosure. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one skill in the art would consider equivalent to the recited values (i.e., having the same function or result). In many instances these terms may include numbers that are rounded to the nearest significant figure. Unless otherwise stated herein, approximately may at least be interpreted as +/−5 mm if expressed in mm and +/−2% if expressed in percentage. In this document, the term “longitudinal” should be understood to mean in a direction corresponding to an elongated direction of the enclosure, or in direction spanning from the opening of the enclosure to the distal tip of the enclosure, wherein “traverse” should be understood to mean in a side-to-side direction or 180°+/−10° relative to the longitudinal direction.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of an absorbent penile enclosure in accordance with one embodiment of the present invention;

FIG. 2 is an elevational front view of the absorbent penile enclosure in FIG. 1 disposed within its packaging in accordance with one embodiment of the present invention;

FIG. 3 is an elevational front view of the absorbent penile enclosure in FIG. 1;

FIG. 4 is an elevational side view of the absorbent penile enclosure in FIG. 1;

FIG. 5 is a cross-sectional view of the absorbent penile enclosure in FIG. 4 and along section line 5-5;

FIG. 6 is a perspective view of an absorbent penile enclosure with a catheter-like tube in accordance with one embodiment of the present invention;

FIG. 7 is a cross-sectional view of the absorbent penile enclosure in FIG. 6;

FIG. 8 is a perspective view of an absorbent penile enclosure in accordance with one embodiment of the present invention;

FIG. 9 is an elevational front view of the absorbent penile enclosure in FIG. 8 disposed within its packaging in accordance with one embodiment of the present invention;

FIG. 10 is an elevational front view of the absorbent penile enclosure in FIG. 8 and with the removable attachment strip in a removed and open position in accordance with one embodiment of the present invention;

FIG. 11 is a cross-sectional view of the absorbent penile enclosure in FIG. 10 and along section line 11-11;

FIG. 12 is a perspective view of an absorbent penile enclosure with a catheter-like tube in accordance with one embodiment of the present invention;

FIG. 13 is a plan view of the inner surface of an absorbent penile enclosure in accordance with one embodiment of the present invention;

FIG. 14 is a plan view of the outer surface of the absorbent penile enclosure in FIG. 13; and

FIGS. 15-18 depict perspective views of the absorbent penile enclosure in FIG. 13 being applied onto a user's penis in accordance with one embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

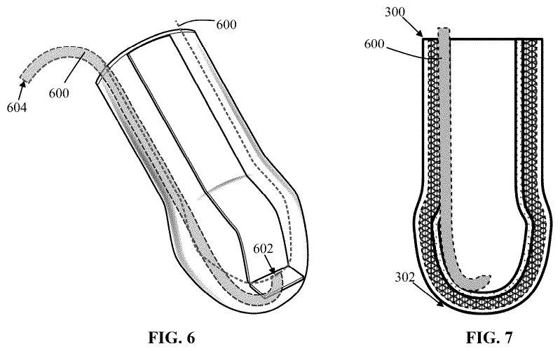

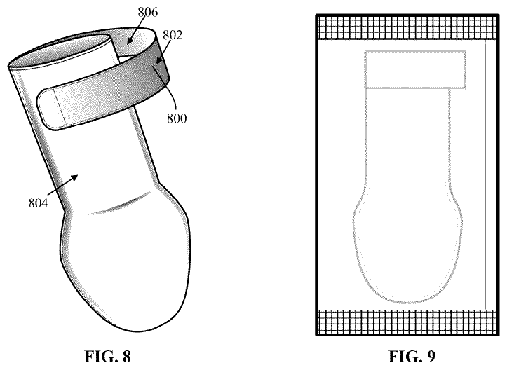

The present invention provides a novel and efficient absorbent penile enclosure configured to effectively and securely absorb urine and other liquids from a user's penis. Referring now to FIGS. 1-5, one exemplary embodiment of the present invention is shown in various views and designed for a child. FIGS. 6-7 depict an exemplary embodiment of the present invention designed for an adolescent or adult. FIGS. 8-12 depict other exemplary embodiments for an adolescent or adult. Regardless the target user, the above-referenced figures show several advantageous features of the present invention, but, as will be described below, the invention can be provided in several shapes, sizes, combinations of features and components, and varying numbers and functions of the components. With reference to FIGS. 1-5 by way example, an absorbent penile enclosure 100 is disclosed. The enclosure 100 may include a body 102 that is formed with multiple layers 500, 502, 504 that are unitary in nature, i.e., operating and functioning as a single or uniform unit, which also includes the enclosure moving and being applied as a single structure. The body 102 is also preferably formed into a cylindrical shape with a bulbous distal end portion (as best seen in FIG. 7) to ensure a more snug fit with the user.

The body 102 may include two opposing and terminal ends 300, 302 that define an enclosure length 304. The enclosure length 304 preferably spans approximately 38-203 mm, or 1.5-6 in so as to provide an adequate and desired length for the user (wherein, beneficially, complete coverage of the user's penis is not required to receive the functional benefit of the enclosure 100). If the enclosure length 304 is too long, it may cause the enclosure to be inadvertently dislodged. Preferably, the enclosure length is approximately 44 mm (+/−5 mm), or 1.8 in (+/−1.6-2.0 in) for child and 91 mm (+/−5 mm), or 3.6 in (+/−0.2 in) for an adolescent or adult. In one embodiment, all three layers 500, 502, 504 span the entire enclosure length 304. In other embodiments, just one of the layers 500, 502, 504 span the enclosure length 304.

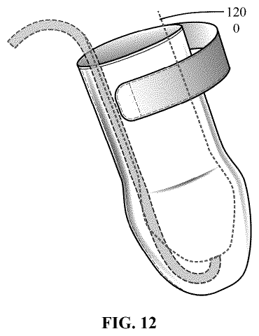

With reference specifically to FIG. 5, the enclosure 100 beneficially includes three layers, an inner layer 500, an intermediate layer 502 superimposed over the inner layer 500, and an exterior layer 504 superimposed over the intermediate layer 502. Each layer may be directly coupled to each other using adhesive or heat. In one embodiment, the inner layer 500 defines an entrance aperture 506 and defines and encloses a cavity 508 that is spatially coupled to the entrance aperture 506, i.e., the user's penis (exemplified with outlines 600, 1200 in FIG. 6 and FIG. 12) is inserted into and through the entrance aperture 506 and disposed into the cavity 508 until reaching or being disposed proximal to an internal distal surface 510. The term “proximal” is defined as located at or near, within 25 mm from, the referencing surface or structure. Said differently, the enclosed entrance aperture 506 is beneficially shaped and sized (e.g., a circular shape having a diameter of approximately 25-100 mm) to provide a single penile pathway into the cavity 508.

The inner layer 500 is beneficially of a permeable material fabric material, wherein said material enclosing the cavity 508. The inner layer 500 is moisture wicking, in that it is configured to transport liquid therethrough. Some exemplary, yet effective, materials for the inner layer 500 include merino wool, bamboo, nylon, or polyester fiber. As the inner layer 500 will directly contact the user's penis, it may beneficially be infused with aloe material. The inner layer 500 has a thickness that is generally less than the intermediate layer 502 and greater than the exterior layer 504.

The intermediate layer 502 is superimposed over and also encloses the inner layer 500, preferably through the entire enclosure length 304 where the inner layer 500 is located. The intermediate layer 502 has an intermediate layer thickness that is greater than the inner layer thickness, as the intermediate layer 502 is designed and configured to retain any emitted liquid. Beneficially, the intermediate layer 502 is made of a fabric absorbent material to retain said liquid and is more absorbent and not as moisture wicking as the inner layer 500.

The exterior layer 504 is also superimposed over and encloses the intermediate layer 502. The exterior layer 504 has an exterior layer thickness that is less than the intermediate layer thickness and less than the thickness of the inner layer 500. The exterior layer 504 is beneficially of an impervious material, thereby preventing the retained liquid in the intermediate layer 502 from escaping the enclosure 100 through the outermost layer. In one embodiment, the exterior layer 504 defines the two terminal opposing ends 300, 302 of the enclosure 100 and principally defines the enclosure length 304. separating the two terminal opposing ends 300, 302 that spans approximately 38-203 mm (1.5-6 in). In one embodiment, the exterior layer 504 is made of a polyethylene material, e.g., a polyethylene film.

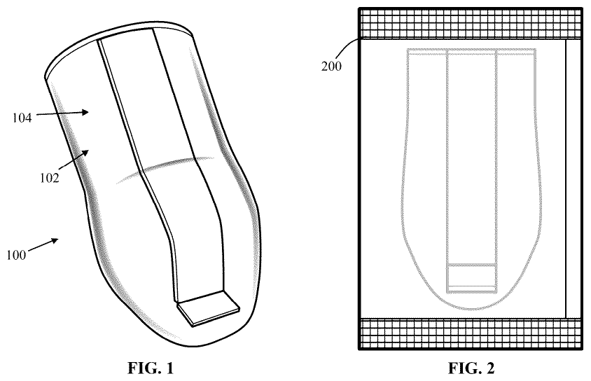

To ensure a lightweight, yet effective, enclosure 100, the inner layer 500 is approximately 0.5-1.5 mm (0.02-0.06 in) in thickness, the intermediate layer 502 is approximately 1-2.5 mm (0.04-0.1 in) in thickness, and the exterior layer 504 is approximately 0.1-0.4 mm (0.004-0.02 in) (preferred is 0.25 mm) in thickness. In preferred embodiments, the thickness of each layer is the same spanning the enclosure length 304. Furthermore, the inner layer 500 and the intermediate layer 502 are also preferably of a thickness less than approximately 3 mm (0.11 in). With reference to FIG. 2, the enclosure 100 is preferably stored in a watertight and/or hermetically sealed package 200 that is configured to be selectively opened by the user before use, thereby preserving the integrity of the adhesive and other contents of the enclosure 100.

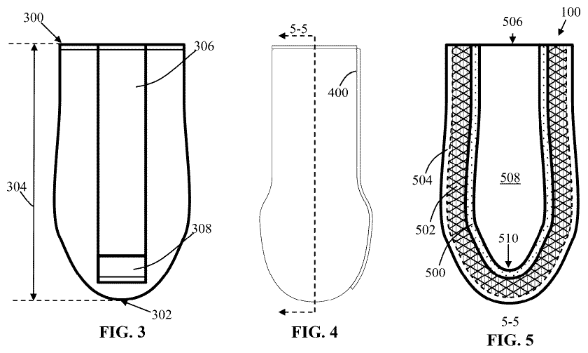

As seen best in FIG. 1, FIG. 3, and FIGS. 6-7, an adhesive attachment film strip 400 is disposed on an outer surface 104 of the exterior layer 504 and substantially spans, i.e., 75% or greater, the enclosure length 304. In other embodiments, the adhesive attachment film strip 400 is located on the inner layer 500 and couplable to a user's penis. When on the outer surface 104, the adhesive attachment film strip 400 beneficially enables the enclosure 100 to adhesively couple to the user's clothing, without damaging the user's clothes and securing the enclosure when the user is more active. To that end, the adhesive attachment film strip 400 is made of a silicone-based or polymer-based material. The adhesive attachment film strip 400 is slender and continuously and substantially spans the enclosure length 304. For example, the adhesive attachment film strip 400 may span 45 mm and be 5-12 mm in width that may be uniform.

The adhesive attachment film strip 400 beneficially includes a release liner 306 superimposed over and coupled to the adhesive film strip 400, is operably configured to be removed from and expose the adhesive film strip 400, and also longitudinally spans substantially the enclosure length 304. The release liner 306 is preferably of a paper material conducive to being removed without removing the adhesive. The release liner 306 may include a removal tab portion 308 uncoupled to the adhesive film strip 400, is operably configured to be removed from and expose the adhesive film strip 400, and that longitudinally spans substantially (75% or greater) the enclosure length 304. The release liner 306 is completely removable from the exterior layer 504 to expose the adhesive strip 400.

As seen best in FIGS. 8-12, the enclosure 100 may also include a removable attachment strip 800 of a flexible material, similar to the material for the exterior layer 504. The attachment strip 800 may be completely removable or, preferably, partially removable relative to the exterior layer 504. The attachment strip 800 includes a first end 1000 coupled (preferably permanently coupled) to an outer surface 804 of the exterior layer 504, a second free end 1002 opposing the first end 1000 of the removable attachment strip 600, and a removable strip length (approximately 25-50 mm) separating the first end 1000 and second free end 1002. The second end 802 is “free” in that it is not permanently coupled to any external structure or surface, and can be selectively removed from the exterior layer 504. The removable attachment strip 800 includes an outer surface 802, an inner surface 806 opposing the outer surface 802 of the removable attachment strip 800, includes an adhesive material (like the adhesive used for the attachment film strip 400) disposed on the inner surface 806 of the removable attachment strip 800, and is operably configured to at least partially surround and adhesively couple to a circumference of the outer surface 804 of the exterior layer 504.

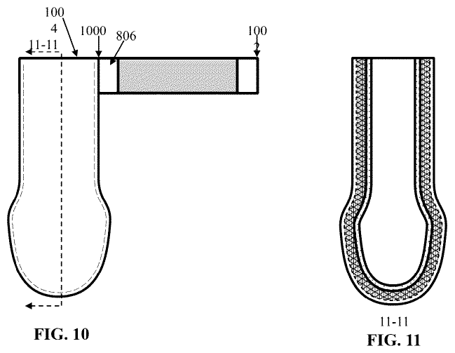

The removable attachment strip 800 also may include adhesive of a silicone-based or polymer-based material and is preferably transversely oriented relatively to the longitudinal orientation of the enclosure (as best seen in FIG. 10). The attachment strip 800 has dimensions akin to the adhesive attachment film strip 400 and beneficially provides a compressive force on the shaft of a user's penis, securing the enclosure thereto. The attachment strip 800 is preferably proximal and transversely oriented to the terminal end 1004 of the enclosure. The outer surface 804 may also include an adhesive material disposed thereon for securing the enclosure to a user's clothes. The adhesive on the attachment strip 800 may be completely disposed thereon, e.g., over the entire surface area, or partially disposed thereon, preferably on a central portion of the surface of the attachment strip 800 (as best seen in FIG. 10).

In one embodiment of the present invention and as best seen in FIGS. 5-7, the enclosure 100 includes a tube 600 defining an entrance aperture 602, defining an exit aperture 604, and a tube channel separating the entrance and exit apertures 602, 604 of the tube 600. The entrance aperture 602 is preferably disposed within the cavity 508, proximal to one of the two terminal opposing ends 300, 302 of the absorbent enclosure 100, and proximal to the inner layer 500 (as exemplified best in FIG. 7). The tube 600 is operably configured to transport liquid from the entrance aperture 602 of the tube 600 through the tube channel and out through the exit aperture 604 of the tube 600. The discharge into the tube 600 may lead to a drainage bag that is selectively drained by the user.

As such, an absorbent penile enclosure is disclosed that safely, effectively, and securely enables liquid discharge to be removed and prevents damage or moisture from reaching the user's clothes. The enclosure includes an adhesive on a side of the enclosure for attachment to a user's clothing or to the user.

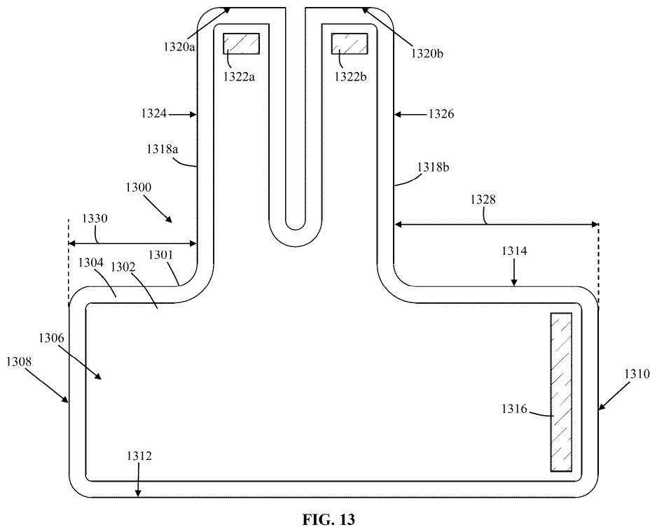

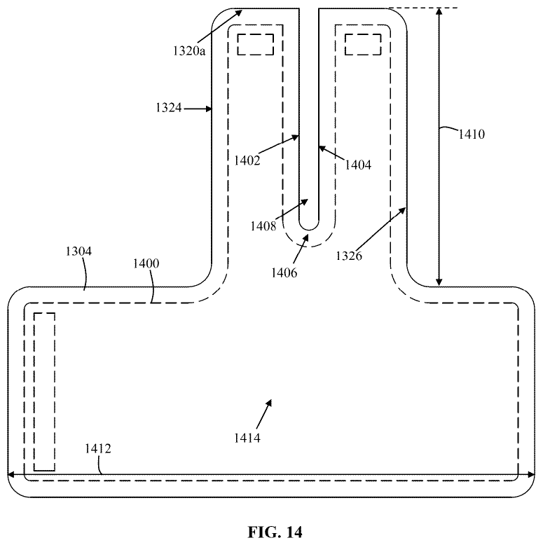

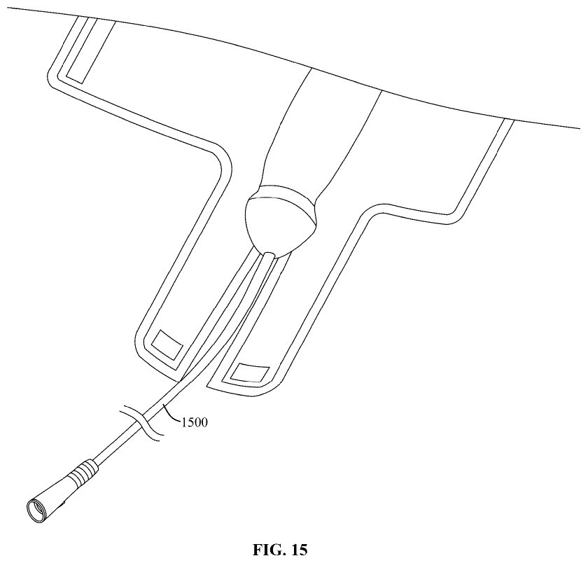

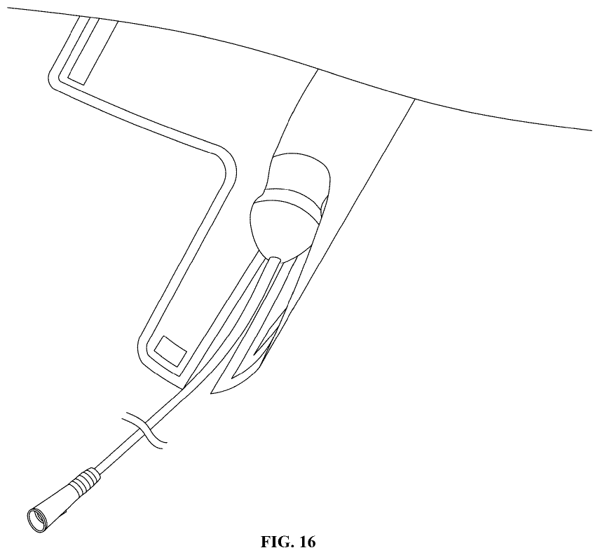

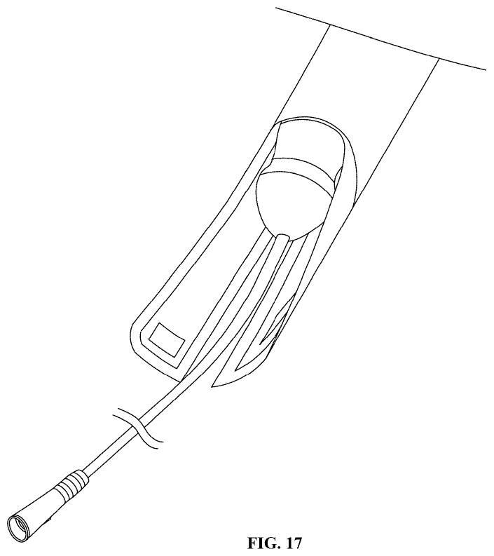

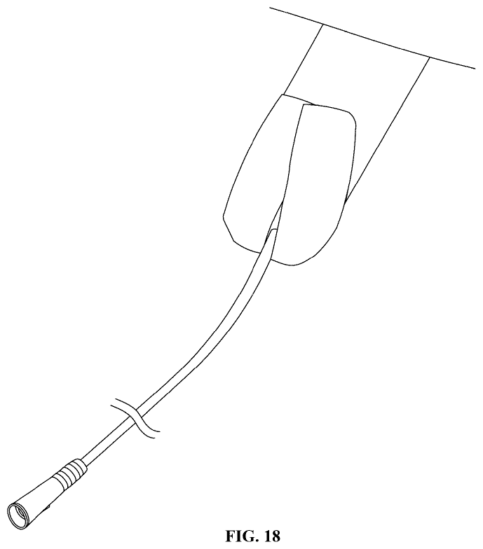

With reference to FIGS. 13-18, another embodiment of the present invention is depicted. In particular, the absorbent penile enclosure 1300 may include a unitary body 1301 with an absorbent layup configuration and material as described above. The body 1301 may be described as being unitary, i.e., it moves a singular unit and is operable to coupled and uncoupled to a user's penis as a singular unit. Furthermore, the absorbent layup may include or consist of an inner layer 1302 of a permeable material fabric material, an intermediate layer (e.g., the intermediate layer 502 discussed above in reference to FIG. 5) superimposed over the inner layer and also of a fabric material that is absorbent, and an exterior layer 1304 superimposed over and enclosing the intermediate layer. With reference specifically to FIG. 14, the layers may be coupled together with adhesive and/or sealed with a threading or welding. For example, the inner layer 1302 may encapsulate the intermediate layer by sealing the exterior layer 1304 around a perimeter of the inner layer 1302 as evidenced by a demarcation line 1400 representing adhesive, pressure seal, threading, etc. The exterior layer 1304 is of an impervious material to inhibit transfer of liquid therethrough.

In one embodiment, the intermediate layer is encapsulated by the inner and exterior layers 1302, 1304 and each layer defines a thickness. In particular, the exterior layer thickness of the exterior layer 1304 is preferably less than an intermediate layer thickness of the intermediate layer 1304 and the intermediate layer thickness is greater than the inner layer thickness of the inner layer 1302. This configuration enables a layup that effectively absorbs and transfers liquid away from the user's penis, while providing a comfortable and discrete product conducive to most users.

Referring back to FIG. 13 by way of example, the unitary body 1301 is configured with a base portion 1306 defined by two opposing outer side edges 1308, 1310 of the unitary body 1301, an outer lower edge 1312, and an outer upper edge 1314. Each of said edges may be formed with the exterior layer 1304 and may be relatively planar with the exception of the corners, which may be rounded or chamfered. The edges 1308, 1310, 1312, 1314, 1324, 1326 may be considered the perimeter edges of the body 1301. As best seen in FIGS. 15-17, the base portion 1306 is configured to surround the user's penis, and may have at least one base fastener 1316 for securing one end of the bae portion 1306 to an opposite end of the base portion 1306 (i.e., on two opposite sides of the two auxiliary coupling members 1318a-b). In one embodiment, the base fastener(s) 1316 is an adhesive (peel and stick and/or PSA), hook and/or loop material, snap, etc.) and is disposed proximal one of the two opposing outer side edges 1308, 1310. The term “proximal” is defined as at, adjacent, or near (within 10% of the overall length 1412 of the base portion 1306) the referring structure.

In one embodiment, the unitary body 1301 includes at least one base fastener 1316 is disposed on the inner layer 1302 and is configured to directly couple with a fastener disposed on the exterior layer 1304. The fastener on the exterior layer shown in the figures is the outer surface 1414 of the exterior layer 1304, namely the hook and/or loop. In another embodiment, the fastener disposed on the exterior layer 1304 may be adhesive, corresponding hook and/or loop, etc.

In one embodiment, the unitary body 1301 includes two auxiliary coupling members 1318a-b that each extend outwardly from the outer upper edge 1314 of the base portion 1306, are disposed in a cantilevered configuration, have a distal edge 1320a-b, have the absorbent layup thereon, and have at least one auxiliary fastener 1322a-b (e.g., adhesive, hook and/or loop material, snap, etc.) disposed proximal to the distal edge 1320a-b. Said another way, the two auxiliary coupling members 1318a-b are flexible portions of the unitary body 1301 that are fixed at one end to the base portion 1306 and free at the opposite end so as to wrap partially or fully around the user's penis (as exemplified in FIG. 18).

In one embodiment, the two auxiliary coupling members 1318a-b each have an outer side edge, e.g., edges 1324, 1326, wherein the base portion 1306 is configured with a first offset length 1328 that may be approximately 1-3 in separating one of the two opposing outer side edges 1308, 1310 and one of the outer side edges 1324, 1326 of the two auxiliary coupling members 1318a-b. The first offset length 1328 is beneficially greater than a second offset length 1330 of approximately 1-3 in separating another of the two opposing outer side edges 1308, 1310 and another of the outer side edges 1324, 1326 of the two auxiliary coupling members 1318a-b. Said another way, the greater of the two offset lengths 1328, 1330 minimizes the material surrounding the user's penis and interference with a catheter, yet providing sufficient enclosure.

In another embodiment, the one or more auxiliary fastener(s) 1322a-b are disposed on the inner layer 1302 and are configured to directly couple with a fastener (e.g., an outer surface, adhesive, corresponding hook and/or loop, etc.) disposed on the exterior layer 1304 on the base portion 1306. One or both of the two auxiliary coupling members 1318a-b may also include inner side edges 1402, 1404 opposing one of the outer side edges 1324, 1326 thereon, wherein the inner side edges 1402, 1404 may be oriented at substantially parallel orientations (+/−5°) with the corresponding outer side edges 1324, 1326 on the two auxiliary coupling members 1318a-b.

In one embodiment, the inner side edges 1402, 1404 define a channel 1408 spanning from a joint 1406 formed by the two auxiliary coupling members 1318a-b to the distal edges 1320a-b thereon. The channel 1408 critically provides the ability for the user to either completely wrap the tip of the penis without the any catheter or similar object or wrap the penis and provide a means for the catheter (including tube) to be inserted therethrough. The channel 1408 is preferably less than 1 inch spanning from the joint 1406 to the distal edges 1320a-n on each of the two auxiliary coupling members 1318a-b. The channel 1408 is also substantially uniform, with the exception off the location proximal to the joint 1406. Beneficially, the joint 1406 is also vertically displaced approximately less than 1 in. from both the corresponding edges 1314 on adjacent sides of the edges 1324, 1326 to also provide the ability for the user to either completely wrap the tip of the penis without the any catheter or similar object or wrap the penis and provide a means for the catheter (including tube) to be inserted therethrough. With reference to FIG. 15, an exemplary catheter tube 1500 is illustrated disposed in the channel 1408 and configured to remove liquid from a user's penis out through the unitary body 1301. When the user desires to remove the body 1301, the user will similarly uncouple the fasteners 1316, 1322a-b and remove the body 1301 for disposal.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above-described features.

What is claimed is:

1. An absorbent penile enclosure comprising:
a unitary body:
with an absorbent layup including an inner layer of a permeable material fabric material, including an intermediate superimposed over the inner layer and of a fabric absorbent material, including an exterior layer superimposed over and enclosing the intermediate layer and of an impervious material;
configured with a base portion defined by two opposing outer side edges of the unitary body, an outer lower edge, and an outer upper edge, having the absorbent layup thereon spanning along the base portion for surrounding a user's penis, and having at least one base fastener disposed proximal to one of the two opposing outer side edges; and
configured with two auxiliary coupling members each extending outwardly from the outer upper edge of the base portion, disposed in a cantilevered configuration with a distal edge, having the absorbent layup thereon, and having at least one auxiliary fastener disposed proximal to the distal edge.

2. The absorbent penile enclosure according to claim 1, wherein the intermediate layer is encapsulated by the inner and exterior layers.

3. The absorbent penile enclosure according to claim 1, wherein an exterior layer thickness of the exterior layer is less than an intermediate layer thickness of the intermediate layer and the intermediate layer thickness is greater than the inner layer thickness of the inner layer.

4. The absorbent penile enclosure according to claim 1, wherein the at least one base fastener is disposed on the inner layer and is configured to directly couple with a fastener disposed on the exterior layer.

5. The absorbent penile enclosure according to claim 4, wherein the two auxiliary coupling members each further comprise:
outer side edges, wherein the base portion is configured with a first offset length separating one of the two opposing outer side edges and one of the outer side edges of the two auxiliary coupling members that is greater than a second offset length separating another of the two opposing outer side edges and another of the outer side edges of the two auxiliary coupling members.

6. The absorbent penile enclosure according to claim 4, wherein the at least one auxiliary fastener is disposed on the inner layer and is configured to directly couple with a fastener disposed on the exterior layer on the base portion.

7. The absorbent penile enclosure according to claim 1, wherein the at least one auxiliary fastener is disposed on the inner layer and is configured to directly couple with a fastener disposed on the exterior layer on the base portion.

8. The absorbent penile enclosure according to claim 1, wherein each of the two auxiliary coupling members further comprise:
inner side edges opposing one of the outer side edges thereon, the inner side edges define a channel spanning from a joint formed by the two auxiliary coupling members to the distal edges thereon.

9. The absorbent penile enclosure according to claim 1, wherein the channel is less than 1 inch spanning from the joint to the distal edges on each of the two auxiliary coupling members.

10. The absorbent penile enclosure according to claim 1, further comprising a catheter tube disposed in the channel and configured to remove liquid from a user's penis out through the unitary body.

11. The absorbent penile enclosure according to claim 1, wherein the inner layer is infused with an aloe material.

12. The absorbent penile enclosure according to claim 1, wherein the inner layer of a polyester fiber material.

13. The absorbent penile enclosure according to claim 1, wherein the exterior layer is of a polyethylene material.

14. The absorbent penile enclosure according to claim 1, wherein the inner layer is of a moisture wicking material.

15. The absorbent penile enclosure according to claim 7, wherein the inner layer and the intermediate layer are of a thickness less than approximately 3 mm.
